(19) United States
(12) Patent Application Publication
(10) Pub. No.: US 2008/0287893 A1
(43) Pub. Date: Nov. 20, 2008

(54) AIR SUCTIONING AND FILTERING DEVICE HAVING INSTANTLY AVAILABLE AIR SUCTIONING AND THERMAL SENSING

(76) Inventor: Leonard Ineson, Mississauga (CA)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/422,008

(22) Filed: Jun. 2, 2006

Related U.S. Application Data
(63) Continuation-in-part of application No. 11/300,386, filed on Dec. 15, 2005, now abandoned.

(51) Int. Cl. A61M 1/00

(52) U.S. Cl. 604/319

(57) ABSTRACT

An air suctioning and filtering device for use in an surgery plume evacuation system comprises a suction inlet, a filter connected in fluid communication with the suction inlet, a connector pipe connected in fluid communication with the filter, and a vacuum fan connected in fluid communication with the connector pipe to suction air into the suction inlet, through the filter, and through the connector pipe. An air by-pass inlet having a shutter valve mounted thereon permits air to be suctioned to the source of suction without ingressing into the suction inlet, when the shutter valve is in a standby position. A much smaller amount of air is suctioned through the auction inlet. In a full suctioning position, with the shutter valve closed over the air by-pass inlet, all of the air is suctioned into the suction inlet from an electrosurgical pencil.

### PARENT CASE TEXT

This application is a Continuation-In-Part of application Ser. No. 11/300,386.

### FIELD OF THE INVENTION

The present invention relates to an air suctioning and filtering devices for use in medical applications, and more particularly to such an air suctioning and filtering device for evacuating surgical plumes generated during electrocautery surgery.

### BACKGROUND OF THE INVENTION

During surgery, the tissue of a patient is cut and/or cauterized by means of an electrosurgical pencil. When the metal tip of the electrosurgical pencil touches the tissue at the surgical site, a high frequency electrical current flows from the electrode to the tissue, thus cutting and/or the tissue.

Due to the cauterizing effect of the electrically conductive metal tip, plumes of smoke are produced, which are typically referred to as surgical smoke, and must be removed. This surgical smoke is offensive in terms of its pungent odour, and is also potentially dangerous to surgeons and other operating room staff in that it contains possible carcinogenic elements, and also potentially transportable viral DNA. Exacerbating this problem, is the fact that such plumes of smoke tend to rise toward the persons involved in the operation. Further, the evacuation of smoke away from the surgical site is vital so that the surgeon's view of the operation site remains as unobscured as reasonably possible.

In order to evacuate smoke from the surgical site, some electrosurgical pencils have an air flow passage with an inlet that either terminates adjacent to the electrically conductive metal tip, or is in fluid communication with the metal tip, and an outlet at the opposite end. Alternatively, a separate smoke evacuator tube has an inlet that terminates near the electrically conductive metal tip of the electrosurgical pencil when clipped on to the electrosurgical pencil, and an outlet at the opposite end. In either case, the outlet is configured to receive and retain thereon a plastic air flow hose that is connected to a source of suctioning that causes air to be drawn from the electrosurgical pencil. The air flow must be sufficient to draw away plumes of surgical smoke, which are subsequently filtered before they reach the source of suctioning by a filter made from ULPA media, sponge foam, charcoal, or other suitable material. Typically, an air flow of about ten cubic feet per minute to about thirty cubic feet per minute is sufficient for use with electrosurgical pencils.

However, in use, the source of suction is not turned on until it is required by the surgeon. A momentary contact switch on the electrosurgical pencil, or a foot operated switch, is pressed to operate the electrosurgical pencil as well as start the vacuum motor in the smoke evacuation system, thereby invoking the source of suction. Unfortunately, there is a delay between the time the momentary contact switch is pressed and the time the air suction actually starts drawing air, and therefore the plume of surgical smoke into the electrosurgical pencil or the smoke tube attachment. This time delay is medically significant since the plume of surgical smoke has time to waft towards the surgeon and others, which is highly undesirable.

A typical electrosurgical pencil and surgery plume filter device, also called a smoke evacuation apparatus, is described in U.S. Pat. No. 6,524,307 issued Feb. 25, 2003 to Holland. This surgery plume filter device is for filtering particulate, gases, harmful microbes and orders suspended in the smoke plume generated during surgery.

A system that attempts to overcome the above discussed delay in the suction of surgical smoke is disclosed in U.S. Pat. No. 5,613,966 issued on Mar. 25, 1997 to Makower et al., this patent discloses a System and Method for Accessory Rate Control. This system changes the rate of operation of the smoke evacuator used with an electrosurgical generator during treatment of a patient electrosurgically at an operative site. A mono or bipolar circuit for electrosurgery has an electrosurgical generator, ESU. A switch in the circuit activates the ESU when keyed. An active output connects the ESU to supply radio frequency energy and a return input to the ESU receives energy. A handpiece has an active electrode and an active lead connects between the output and the electrode. A return lead connects the return input and has a terminus to the patient. A controller in the ESU controls the various output signals of the ESU in response to hand or foot switching inputs or sensed current in the patient circuit. A trigger is connected to the controller in the ESU. The smoke evacuator operates at a different rate when the trigger in the ESU is keyed. A rate controller adjusts the condition of its function per unit time in accord with the trigger on the ESU. Terminals on the ESU deliver signals from the trigger through connections between the terminals and the smoke evacuator. The smoke evacuator senses the signal and changes the rate controller from a low level of fluid flow to a high flow for drawing fluid from the area about the active electrode. The rate controller adjusts. The switch is located on the handpiece or a foot pedal. A method for changing the rate of operation of a smoke evacuator has steps including having a circuit for electrosurgery associated with the ESU. The rate of the smoke evacuator is adjusted with the keying of the ESU or with sensed current from the patient circuit. As can be readily seen, the smoke evacuator as taught in U.S. Pat. No. 5,613,966 to Makower et al. has an inherent delay in the increase in suctioning that is available while it increases in speed, which is highly undesirable.

It is an object of the present invention to provide an air suctioning and filtering device for evacuating a smoke plume generated during electrocautery surgery, which air suctioning and filtering device overcomes the inherent time delay between starting the suctioning and the onset of the suctioning at the electrosurgical pencil.

### SUMMARY OF THE INVENTION

In accordance with one aspect of the present invention there is disclosed a novel air suctioning and filtering device for use in an surgery plume evacuation system. The air suctioning and filtering device comprises an air suction inlet for permitting air to be received from an electrosurgical tool. An air filter means is connected in fluid communication with the air suction inlet for receiving suctioned air therefrom. A conduit means is connected in fluid communication with the filter for receiving filtered air therefrom. A source of suction is connected in fluid communication with the conduit means for suctioning air into the suction inlet, through the air filter means, and through the conduit means. There is an air by-pass inlet on the conduit to permit air to be suctioned to the source of suction without being received from the electrosurgical tool through the air suction inlet. A valve means is operatively mounted on the air by-pass inlet for movement between a standby position, where as approximately two-thirds of the air is suctioned into the air by-pass inlet and the other one-third from the source of suction. In the full suctioning position, the air is precluded from being suctioned into the air by-pass inlet and all air flows from the source of suction. There is also a manually operable switch on the ESU pencil for controlling the valve means. In use, the valve means is moved by operation of the manually operable switch means between its standby position and its full suctioning position. In the standby position, a substantial portion of the air suctioned by the source of suction is suctioned into the air by-pass inlet and a significantly lesser portion of the air suctioned by the source of suction is suctioned into the suction inlet. In the full suctioning position, all of the air suctioned by the source of suction is suctioned into the suction inlet.

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter of which is briefly described herein below.

### BRIEF DESCRIPTION OF THE DRAWINGS

The novel features which are believed to be characteristic of the air suctioning and filtering device according to the present invention, as to its structure, organization, use and method of operation, together with further objectives and advantages thereof, will be better understood from the following drawings in which a presently preferred embodiment of the invention will now be illustrated by way of example. It is expressly understood, however, that the drawings are for the purpose of illustration and description only, and are not intended as a definition of the limits of the invention. In the accompanying drawings:

FIG. 1 is a simplified overall pictorial representation of a preferred embodiment of the air suctioning and filtering device according to the present invention, in use with a surgery plume evacuation system;

FIG. 2 is a perspective view from the front of the preferred embodiment of the air suctioning and filtering device of FIG. 1;

FIG. 3 is a side elevational view of the preferred embodiment air suctioning and filtering device of FIG. 2;

FIG. 4 is a top plan view of a preferred embodiment of the air suctioning and filtering device according to the present invention, with the outer casing removed;

FIG. 5 is a side elevational view of the preferred embodiment air suctioning and filtering device shown in FIG. 2, with the shutter valve of the air suctioning and filtering device in its standby position;

FIG. 6 is an enlarged perspective view from the top of the air by-pass inlet with the shutter valve in a standby position;

FIG. 7 is a top plan view similar to FIG. 6, but with the shutter valve in full suctioning position; and,

FIG. 8 is an enlarged side view of the of the solenoid and shutter valve.

### DETAILED DESCRIPTION OF A PREFERRED EMBODIMENT

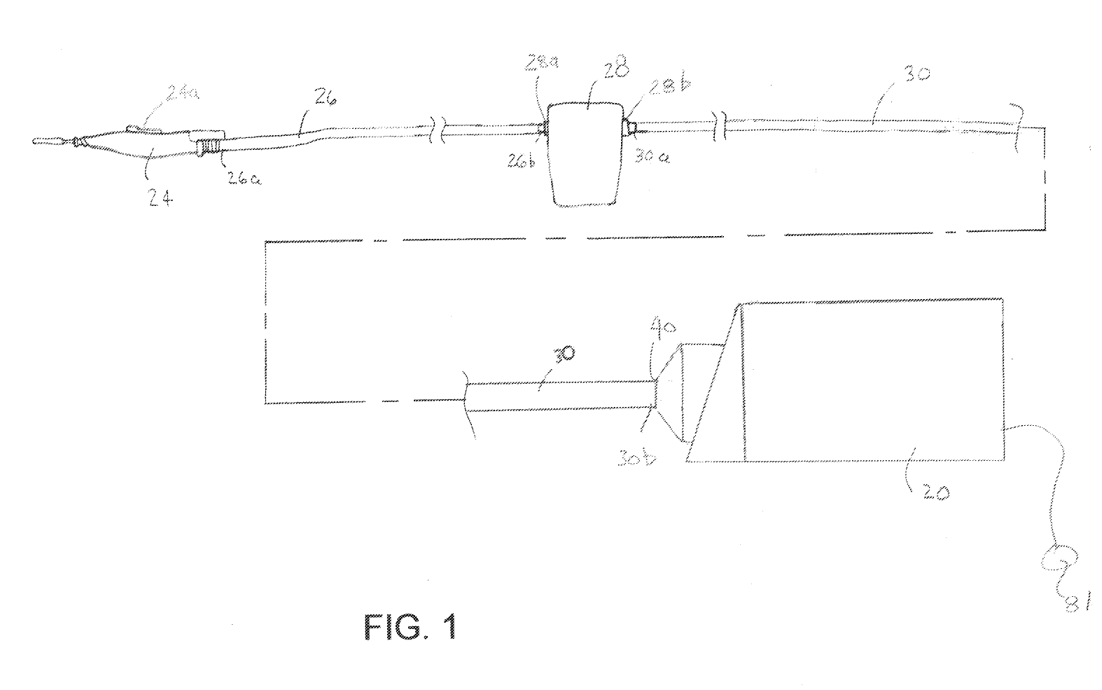

Reference will now be made to FIGS. 1 through 8, which show a preferred embodiment of the air suctioning and filtering device of the present invention, as indicated by general reference numeral 20. The air suctioning and filtering device 20 is for use in an surgery plume evacuation system, as indicated by the general reference numeral 22 in FIG. 1. The surgery plume evacuation system 22 includes an electrosurgical pencil 24, a first flexible tube 26, a suction collection container 28 (which is optional and may or may not be used), a second flexible tube 30, and the air suctioning and filtering device 20. The intake end 26a of the first flexible tube 26 is connected to the electrosurgical pencil 24 and the delivery end 26b of the first flexible tube 26 is connected to the inlet nozzle 28a of the suction collection container 28 to permit delivery of air, smoke, moisture, liquid, and particulate matter from the electrosurgical pencil 24 to the suction collection container 28. The intake end 30a of the second flexible tube 30 is connected to the outlet nozzle 28b of the suction collection container 28 and the delivery end 30b of the second flexible tube 30 is connected to a suction inlet 40 of the air suctioning and filtering device 20.

The air suctioning and filtering device 20 is for use in an surgery plume evacuation system 22. The air suctioning and filtering device comprises a suction inlet 40 for permitting air to ingress, as received from the electrosurgical pencil 24.

An air filter means 50 is connected in fluid communication with the suction inlet 40 so as for receiving suctioned air therefrom. The air filter means 50 preferably comprises a suitable air filter, such as a foam filter, a charcoal filter, or other suitable type of filter. The air filter 50 traps moisture, liquid, and particulate matter, and precludes them from travelling downstream. Preferably, the air filter 50 comprises a removable filter within an outer housing 52 mounted on a base 54.

A conduit means comprising a connector pipe 60 is connected in fluid communication with the air filter 50 for receiving filtered air therefrom. The connector pipe 60 is preferably a rigid plastic or metal pipe and about the same diameter as the suction inlet 40, although the exact diameter is not overly important.

A source of suction is connected in fluid communication with the connector pipe 60 to suction air into the suction inlet 40, through the filter means, and through the connector pipe 60. In the preferred embodiment illustrated, the source of suction comprises an electrically powered vacuum fan 70.

Preferably, the suction inlet 40, the air filter means 50, the connector pipe 60 and the vacuum fan 70 are substantially co-linearly aligned to maximize air flow therethrough.

There is an air by-pass inlet 62 on the connector pipe 60 to permit air to be suctioned to the source of suction without ingressing into the suction inlet 40. The air by-pass inlet 62 is located at the top end 69 of a vertically oriented pipe portion 68 extending upwardly from the connector pipe 60. Other suitable positioning and arrangements of the connector pipe 60, the vertically oriented pipe portion 68, and the air by-pass inlet are also permissible.

A valve means 64 is operatively mounted on the air by-pass inlet 62 for movement between a standby position, as is best seen in FIGS. 5 and 6, and a full suctioning position. When the valve means 64 is in its standby position, as best seen in FIG. 7.

In the preferred embodiment, as illustrated, the valve means 64 comprises a shutter valve 64 pivotally mounted at the air by-pass inlet 62. The shutter valve 64 is moved by an electrically powered solenoid 66 connected to the main circuit board 84. When the solenoid 66 is not energized, the shutter valve 64 is open and is in its standby position. When the solenoid 66 is energized, the shutter valve 64 closes and moves to its full suctioning position.

There is a manually operable switch means 80 for controlling the shutter valve 64. The manually operable switch means 80 comprises a switch 24a located on the electrosurgical pencil 24 and a foot operable air switch 81. Both the switch 24a and the foot operable air switch 81 are connected in parallel to an electronic sensor 82 on the main circuit board 84. In use, the shutter valve 64 is moved between its standby position and its full suctioning position by means of operation of the switch 24a on the electrosurgical pencil 24 or the foot operable air switch 81.

In the standby position of the shutter valve 64, a substantial portion of the air suctioned by the source of suction is suctioned into the air by-pass inlet 62 and a significantly lesser portion of the air suctioned by the source of suction is suctioned into the suction inlet 40. In the full suctioning position, substantially all of the air suctioned by the source of suction is suctioned into the suction inlet 40. Particulars will now be discussed in greater detail.

When the shutter valve 64 is in the standby position, it has been found that a suctioning rate of about ten to eleven cubic feet per minute by the vacuum fan 70 creates an airflow of about three to four cubic feet per minute into the suction inlet 40, and therefore at the electrosurgical pencil 24. This slight air flow essentially keeps air moving into the electrosurgical pencil 24 without causing purposeful suctioning. However, this slight air flow is sufficient to allow for immediate full suctioning air flow, when the shutter valve 64 is moved to its full suctioning position. The difference, about seven cubic feet per minute of air flow is suctioned through the suction inlet 40, instead of through the electrosurgical pencil 24, but is available instantly when the shutter valve 64 is moved to its full suctioning position.

In the full suctioning position, air is substantially precluded from being suctioned into the air by-pass inlet 62 and to the vacuum fan 70. Instead, all of the air suctioned by the vacuum fan 70, about ten to eleven cubic feet per minute, is suctioned from the electrosurgical pencil 24 and into the suction inlet 40 of the air suctioning and filtering device 20.

Further, subsequent to the shutter valve 64 moving to its full suctioning position, the vacuum fan 70 is increased from its initial suctioning rate of about ten to eleven cubic feet per minute, to a higher suctioning rate of air flow. It has been found that a rate of suctioning of about twenty to thirty cubic feet per minute by the vacuum fan 70, through the electrosurgical pencil 24, can be achieved when the shutter valve 64 is in its full suctioning position. This volume of air flow is limited by the small inside diameter of the first flexible tube 26 and the second flexible tube 30 (about ¼″ to about ⅜″). The vacuum fan 70 would normally draw about eighty to one hundred five cubic feet per minute of air.

In other words, during actual use, a high suctioning rate of the vacuum fan 70 is desirable for suctioning air through the electrosurgical pencil 24. However, between periods of use, a lesser air flow is sufficient to keep a small amount of air suctioning into the electrosurgical pencil 24. It must be understood though that if the vacuum fan 70 is kept at a initial suctioning rate of just enough to suction a small amount of air (three to four cubic feet per minute) into the electrosurgical pencil 24, the vacuum fan 70 will take significantly longer to change from that low initial suctioning rate to the necessary higher suctioning rate.

Of course, both of the initial suctioning rate and the higher suctioning rate are fully adjustable and could even be the same.

The air suctioning and filtering device 20 further comprises control circuitry 86 for controlling the speed of the initial suctioning rate of the vacuum fan 70, and control circuitry 88 for controlling the speed of the higher suctioning rate of the vacuum fan 70. This control circuitry 86,88 is found on the main circuit board 84.

In standby mode, approximately three to four cubic feet per minute of air (dependent on settings) flows in through the suction inlet 40 from the electrosurgical pencil 24, through the first flexible tube 26, through the suction collection container 28, through the second flexible tube 30, and to the vacuum fan 70 of the air suctioning and filtering device 20. At the same time, approximately seven cubic feet per minute of air flows in through the open shutter valve 64 and on to the vacuum fan 70. At the instant the shutter valve 64 goes into its full suctioning position, a signal is sent to the solenoid 66 which in turn closes the shutter valve 64. All of the air is then suctioned through the suction inlet 40. At the same moment the signal is sent to the solenoid 66, a signal is also sent to the vacuum fan 70 to indicate at which speed the motor will now operate. In most cases, the motor speed will be increased substantially creating greater plume capture at the suction inlet 40.

The suctioning and filtering device 20 is always in standby mode until the electrosurgical pencil 24 is activated or similarly the foot switch 81 is depressed. As a result of this set up, low air flow is always present, creating vacuum in the electrosurgical pencil 24, the first flexible tube 26, the suction collection container 28, the second flexible tube 30, and vacuum fan 70. At the instant the solenoid 66 is activated, thereby moving the shutter valve to its full suctioning position, the air by-pass inlet 62 closes and increased air flow is created. There is no need to create a vacuum in the electrosurgical pencil 24, the first flexible tube 26, the suction collection container 28, the second flexible tube 30, as it was created in the standby mode.

In the present invention, the air suctioned through the air inlet 40, the filter 50 and the connector pipe 60 is used to cool the motor of the vacuum fan 70. However, while the vacuum fan 70 would typically draw about sixty to about eighty five cubic feet of air per minute, it draws significantly less in the present invention due to the reduced diameter of the first flexible tube 26 and the second flexible tube 30, which each have an inside diameter of ¼″. Typically, the electric fan 70 would be hooked up to a tube or a pipe having an inside diameter of ⅞″. Accordingly, it has been found that the electrically powered motor of the vacuum fan 70 can tend to overheat during ongoing use. While the vacuum fan 70 has a built-in thermal sensing unit that shuts down before it actually overheat, it is unacceptable to actually have the vacuum fan 70 shut down during use, as this would stop the air suctioning.

In order to overcome this problem, the present invention also comprises a thermal sensor 90 mounted adjacent the vacuum fan 70 for sensing the ambient temperature of the vacuum fan 70. The thermal sensor 90 is also operatively connected to the electrically powered solenoid 66 through control circuitry 89 on the main circuit board 84. The thermal sensor 90 causes the electrically powered solenoid to be energized when the thermal sensor 90 is above a threshold temperature. A threshold temperature of about 160° F. to about 170° F. has been found to be suitable. At the threshold temperature, the thermal sensor 90 signals the control circuitry 89 to de-energize the electrically powered solenoid 66, thereby moving the shutter valve 64 back to the open standby position, thus permitting a high volume of air to flow through the air by-pass inlet 62 and to the vacuum fan 70. In this manner, the motor of the vacuum fan 70 is cooled without the air suctioning and filtering device 20 shutting down and without altering the air flow of the electrosurgical pencil 24 by any significant amount. It has been found that opening the air by-pass inlet 62 with the vacuum fan 70 at its higher suctioning rate.

As can be understood from the above description and from the accompanying drawings, the present invention provides an air suctioning and filtering device for evacuating a smoke plume generated during electrocautery surgery, which air suctioning and filtering device overcomes the inherent time delay between starting the suctioning and the onset of the suctioning at the electrosurgical pencil, all of which features are unknown in the prior art.

Other variations of the above principles will be apparent to those who are knowledgeable in the field of the invention, and such variations are considered to be within the scope of the present invention. Further, other modifications and alterations may be used in the design and manufacture of the suction collection container of the present invention without departing from the spirit and scope of the accompanying claims.

I claim:

1. An air suctioning and filtering device for use in an surgery plume evacuation system, said air suctioning and filtering device comprising:
an air suction inlet for permitting air to be received from an electrosurgical tool;
an air filter means connected in fluid communication with said air suction inlet for receiving suctioned air therefrom;
conduit means connected in fluid communication with said filter for receiving filtered air therefrom;
a source of suction connected in fluid communication with said conduit means for suctioning air into said suction inlet, through said air filter means, and through said conduit means;
an air by-pass inlet on said conduit means to permit air to be suctioned to said source of suction without being received said electrosurgical tool through said air suction inlet;
valve means operatively mounted on said air by-pass inlet for movement between a standby position, whereat air is suctioned into said air by-pass inlet and to said source of suction, and a full suctioning position, whereat air is substantially precluded from being suctioned into said air by-pass inlet and to said source of suction;
manually operable switch means for controlling said valve means;
wherein, in use, said valve means is moved by operation of said manually operable switch means between its standby position, such that a substantial portion of the air suctioned by said source of suction is suctioned into said air by-pass inlet and a significantly lesser portion of the air suctioned by said source of suction is suctioned into said suction inlet, and its full suctioning position, such that substantially all of the air suctioned by said source of suction is suctioned into said suction inlet.

2. The air suctioning and filtering device of claim 1, wherein said air filter means comprises a removable filter within an outer housing.

3. The air suctioning and filtering device of claim 1, wherein said suction inlet, said air filter means, said connector pipe and said source of suction are substantially co-linearly aligned to maximize air flow therethrough.

4. The air suctioning and filtering device of claim 1, wherein when said valve means is in its standby position, said source of suction is at a initial suctioning rate, and when said valve means is in its full suctioning position, said source of suction is at a higher suctioning rate.

5. The air suctioning and filtering device of claim 4, wherein said source of suction comprises an vacuum fan.

6. The air suctioning and filtering device of claim 5, further comprising control circuitry for controlling the speed of said initial suctioning rate of said vacuum fan.

7. The air suctioning and filtering device of claim 6, further comprising control circuitry for controlling the speed of said higher suctioning rate of said vacuum fan.

8. The air suctioning and filtering device of claim 1, wherein said valve means comprises a shutter valve pivotally mounted at said air by-pass inlet.

9. The air suctioning and filtering device of claim 8, wherein said shutter valve is moved by an electrically powered solenoid.

10. The air suctioning and filtering device of claim 9, wherein said manually operable switch means comprises a foot operable air switch connected to an electronic sensor.

11. The air suctioning and filtering device of claim 9, wherein said source of suction comprises an vacuum fan, and further comprising a thermal sensor mounted adjacent said vacuum fan and operatively connected to said electrically powered solenoid for causing said electrically powered solenoid to be energized when said thermal sensor is above a threshold temperature.

12. The air suctioning and filtering device of claim 9, wherein said conduit means comprises a connector pipe.
